(19) United States
(12) Patent Application Publication
(10) Pub. No.: US 2025/0241553 A1
(43) Pub. Date: Jul. 31, 2025

(54) ADVANCED WEARABLE HYDRATION MONITORING SYSTEM

(71) Applicant: Predictive Wear, Lafayette, IN (US)

(72) Inventors: Matthew Albaugh, Lafayette, IN (US); Orlando Hoilett, Lafayette, IN (US); Axel Masquelin, Lafayette, IN (US); Michael Drakopoulos, Lafayette, IN (US)

(73) Assignee: Predictive Wear, Lafayette, IN (US)

(21) Appl. No.: 19/038,700

(22) Filed: Jan. 27, 2025

Related U.S. Application Data
(60) Provisional application No. 63/625,391, filed on Jan. 26, 2024.

(51) Int. Cl. A61B 5/0537; A61B 5/00; A61F 13/08

(52) U.S. Cl. CPC A61B 5/0537; A61B 5/0022; A61B 5/6804; A61F 13/08; A61B 2560/0214; A61B 2562/0209; A61B 2562/0219; A61B 2562/04

(57) ABSTRACT

A system and method for hydration monitoring of a user with compression garment material and a plurality of sensors, an impedance monitor having a multiplexed, and a multi-frequency analyzer, which analyses several sections of tissue across the wearer in a sequence. The hydration status is provided to a user in real-time, wherein the system can further generate a predictive output of future performance based on current hydration status.

### CROSS-REFERENCE TO RELATED APPLICATION

This U.S. patent application claims priority to U.S. Provisional Application No. 63/625,391 filed Jan. 26, 2024, to the above-named inventors, the disclosure of which is considered part of the disclosure and appendix of this application and is hereby incorporated by reference in its entirety.

### FIELD OF THE INVENTION

The invention relates generally to an apparatus, method, and system for providing hydration status on an individual. More particularly, the invention of the present disclosure relates to a wearable hydration monitoring system.

### BACKGROUND

Monitoring the hydration status of athletes is pivotal in physical performance, recovery, and health, especially during rigorous activities or in hot conditions. Dehydration can lead to impaired performance and an increased risk of exertional heart illness, such as heat exhaustion and heat stroke.

The current wearable landscape consists of specialized devices focusing on specific metrics. There is currently a lack of real-time monitoring as many existing techniques to determine hydration status, such as measuring body weight changes, urine indices (color, osmolality, specific gravity), blood markers (plasma osmolality), and thirst perception, do not offer real-time results. Some methods, such as blood testing, are invasive and complex. Also, relying on thirst perception, urine color is too subjective. Moreover, most of the available methods are not portable because they require heavy stationary equipment.

Most wearable technologies tend to be limited to a specific type of garment or accessory, such as wristbands, watches, or chest straps. Most wearables provide real-time data but lack sophisticated predictive capabilities. Very few wearables provide hydration status predictions, and those that do typically rely on indirect metrics, such as sweat rate, or require manual input of water intake from the user. The feedback from existing wearables is typically limited to visual displays on a screen or basic haptic feedback. Most wearables have associated mobile applications with limited features, primarily focusing on displaying the data. While some devices offer heart rate tracking, very few allow cardiac performance metrics.

Bioimpedance vector analysis (BIVA) is a measurement of complex impedance, normalized for a subject's height. Hydration status may be obtained by comparing the subject's BIVA pattern relative to other individuals with the same race, age, body mass index and sex. Thousands of subjects have been studied to validate this phenomena and tolerance ellipses have been constructed for the BIVA measurement at 50 kHz and deviation outside of the 75% tolerance ellipse represents abnormal tissue impedance. or elite athletes and soldiers, this comparison is less useful as their typical body composition profile and activity deviates them well outside of the standard population.

Consequently, studies have been conducted to develop population specific tolerance ellipses for these specific populations. In addition to comparing an individual's BIVA profile to a population's profile, it is useful to analyze an individual's BIVA profile over time as it may reveal insights into that individual's hydration baseline and their deviation from that baseline while becoming dehydrated. An individual's BIVA profile has been shown to correlate significantly to their race performance in an ironman triathlon. Interestingly, it has been noted that a mitigating factor of BIVA, which typically utilizes one frequency only, is that it cannot accurately estimate both intra and extracellular fluid levels in the user.

It would be advantageous to have an improved device that can provide an integrated, holistic approach, ensuring wearers receive a comprehensive insight into their physiology, hydration status, and athletic performance. Furthermore, it would be advantageous to have a device that can provide a more accurate, convenient, and real-time method for monitoring hydration status on an individual. Still further, it would also be advantageous to have a device that can be wearable by the user. The apparatus of the present disclosure can fulfill an unmet need by providing a device and system for real-time and non-invasive monitoring of hydration status and other biological data using bioelectrical impedance analysis (BIA), enabling athletes, coaches, or individuals to make immediate adjustments in their hydration practices.

### BRIEF SUMMARY OF THE INVENTION

In one aspect, a wearable hydration monitoring apparatus of the present disclosure can continuously monitor how well-hydrated the wearer is by measuring one or more of the body's electric properties. The apparatus can obtain, in real-time, information regarding whether the wearer is drinking enough water or if the wearer needs to re-hydrate. In one exemplary embodiment, the apparatus may comprise a wearable impedance monitor that integrates seamlessly with various garments and accessories. The apparatus can include an impedance monitoring system that can provide direct insights into hydration levels using bioimpedance analysis. The impedance monitoring system can provide a more accurate and timely method than indirect metrics, such as sweat rate, or manual input of water intake from the user. This ensures accurate and timely updates on the wearer's hydration status, prompting the wearer to hydrate as necessary.

The apparatus may be made of various designs, allowing integration with a plethora of wearables. This includes arm sleeves and leg sleeves, undergarments, including underwear and bras, shirts, pants, leggings/spats, shorts, watches or similar wrist-worn devices, athletic shoes, and combinations of the same.

The apparatus is versatile, designed to integrate with a wide array of garments included but not limited to arm sleeves, leg sleeves, underwear, bras, shirts, shoes, and watches. This ensures the wearers can choose a garment that aligns with their comfort and activity type.

The apparatus and system encompass more than typical metrics. It is capable of capturing standard data, such as heart rate, and also capable of measuring impedance, electromyography (EMG), electrocardiogramascope, magnetometer readings, locomotion, activity, body orientation, sleep, and anthropomorphic data. This enables a holistic understanding of the wearer's physiological state.

The apparatus and system can include one or more advanced algorithms to predict a myriad of variables, from hydration status to detailed athletic performance metrics, such as velocity bands, power output, and exercise load. These predictions can aid in optimizing the performance of the wearer and avoiding potential health issues.

The apparatus and system may comprise machine leaning models to predict hydration status on the collected data to provide a more accurate and tailored reading than generic measurement tools.

The apparatus and system provide a multi-modal feedback system, alerting wearers about their hydration status or performance predictions through haptic vibrations, sound or push notifications, enabling more immediate responses from the wearer. The apparatus and system can be coupled to a mobile device application and/or a comprehensive web application via any suitable means including a transceiver or other wireless communication network. The apparatus can be used by coaches and athletic trainers to monitor athletes. The dual platform approach ensures that data is accessible, analyzable, and actionable for both individual users and teams. Additionally, the system can measure heart rate, and also predict advanced cardiac metrics, such as stroke volume and cardiac output, offering a more in-depth insight.

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

FIG. 1 is an illustration wiring diagram of one or more of the electrodes of an exemplary embodiment of an apparatus of the present disclosure.

FIG. 2 is an illustration of the approximate location of electrodes for an arm only application of an exemplary embodiment of an apparatus of the present disclosure, wherein the outermost electrodes can be current injecting, and the innermost two electrodes are target or sensing electrodes. More or less electrodes can be utilized as necessary to allow one or more injecting electrodes and one or more sensing electrodes.

FIG. 3 is an illustration of the approximate location of electrodes for the leg study, wherein the furthest distal electrodes are current injecting and the most proximal two electrodes are Target B.

FIG. 4A is a graphical illustration of a pre-determined hydration threshold as one embodiment of the present disclosure, showing Osmolality higher than 295 indicates dehydration

FIG. 4B is a graphical illustration of a pre-determined hydration threshold as one embodiment of the present disclosure, showing Osmolality higher than 295 indicates dehydration.

FIG. 5 is an illustration of an exemplary embodiment of a central hardware flowchart. Impedance to determine the hydration status of a user can be saved and transmitted to a mobile application device.

FIG. 6 is an illustration of a mobile application flowchart of an exemplary embodiment of a system of the present disclosure.

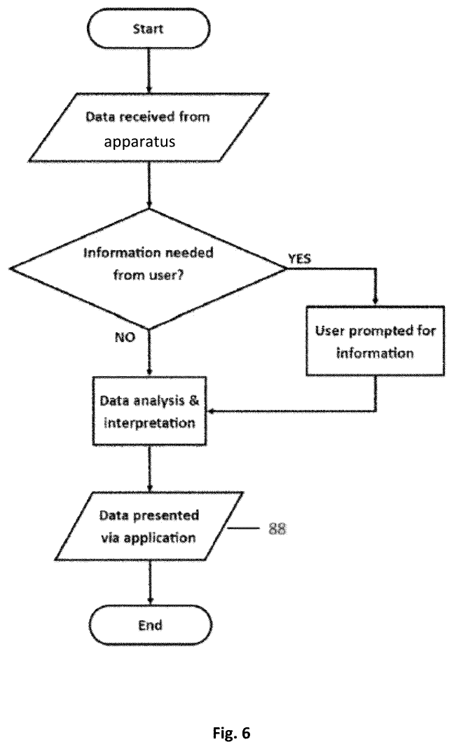

FIG. 7 is an illustration of an exemplary embodiment of a system of the present disclosure utilizing a Recurrent Neural Network (RNN).

### DETAILED DESCRIPTION OF THE INVENTION

The following are more detailed descriptions of various related concepts related to, and embodiments of, methods and apparatus according to the present disclosure. It should be appreciated that various aspects of the subject matter introduced above and discussed in greater detail below may be implemented in numerous ways, as the subject matter is not limited to any particular manner of implementation. Examples of specific implementations and applications are provided primarily for illustrative purposes.

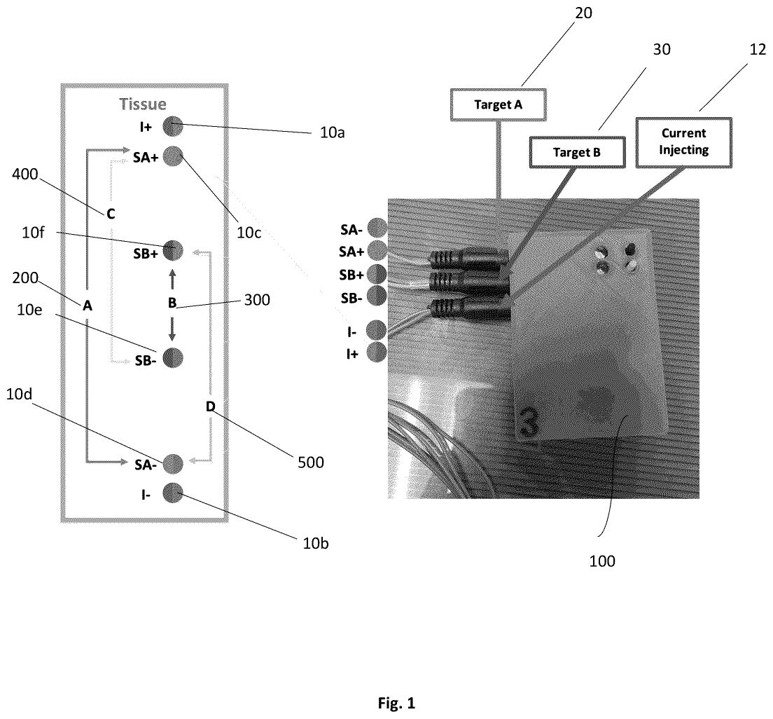

Referring now to the FIGS. 1-4, a hydration monitoring apparatus and system according to the present disclosure and generally referred to herein as the apparatus can be capable of measuring impedance, offering a direct insight into the wearer's hydration levels of a wearer. The apparatus can include an impedance monitor system that can have a multiplexed, and a multi-frequency analyzer, which can analyze several sections of tissue across the wearer in sequence. The apparatus can include any suitable processing means, including but not limited to an on-board 2R1C circuit to benchmark chip performance and provide a basis for calibrating board and wearer specific variances.

The apparatus can include one or more electrodes that can be on the same side, as will the blue electrodes as shown in FIG. 1 The electrodes can take multiple orientation configuration with respect to the anatomy of the subject. I+ could be at the wrist and I− at the upper shoulder or vice versa.

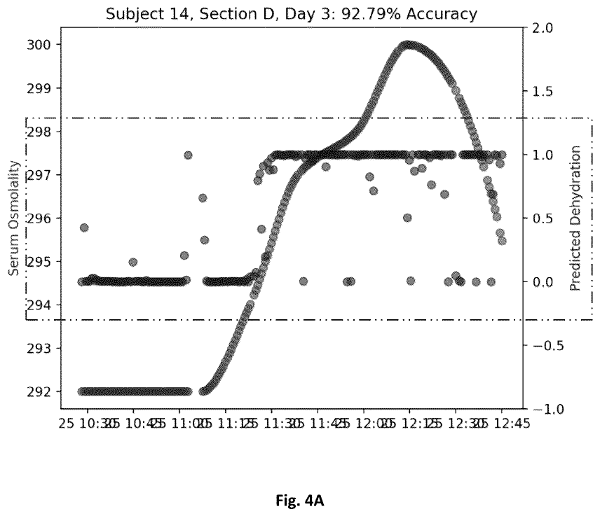

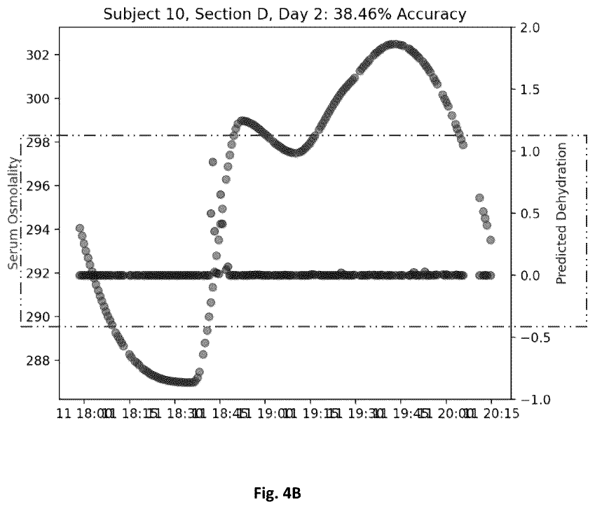

Additionally, the apparatus of the present disclosure can include an on-board potentiometer to calibrate the apparatus prior to each tissue impedance sweep. The apparatus uses high-precision, internal components to calibrate the apparatus for each measurement, ensuring optimal accuracy. The apparatus may comprise an internal peak finder to prevent impedance estimation errors and additionally utilize an attached haptic module to provide the wearer with haptic feedback if the wearer's defined hydration thresholds, heart rate thresholds, or other measurement thresholds have been reached. The user can set one or more pre-determined hydration thresholds and a haptic feedback pattern or notifications. As one exemplary embodiment, a user may wish to be notified when the system determines their likelihood of dehydration is 80% with a single vibration and two vibrations if the likelihood of dehydration exceeds 95% (FIGS. 4A-B). The system can provide an alert to a display to present a color representing a hydration level. In some embodiments, the colors can be green, orange, and red. Green can correspond to an acceptable hydration range reading, orange for a low hydration range reading, and red for an extremely low hydration rand reading. The pre-determined ranges can be used to indicate a wearer's hydration level and similarly indicate a percentage of confidence in a wearer's dehydration.

The apparatus can collect data and detect when the wearer has met a particular pre-determined threshold, or the apparatus collects data, transmits the data to a tertiary apparatus via a wireless or wired connection, which then in turn transmits the data to a backend API. The API can detect when the wearer has met a particular threshold and initiates a process to notify the wearer via the wearer desired mode of notification. In addition, the data may be provided to athletic trainers, coaches, or other interested people who are involved with the wearer's sports team or personal athletic performance interests. The data is provided in a dashboard for the interested person to review in conjunction with any other athletics on the team, if applicable.

Alternatively, the transformer model architecture may generally be fed to each tissue section into its own self attention block and each study arm is trained in parallel. The system can further utilize and obtain multimodal measurements. In addition to impedance, the system may capture a wide array of other physiological metrics such as: heart rate, respiratory rate, blood oxygen, electromyography readings (EMG), electrocardiogramgs (ECG), locomotion, activity level, body orientation, sleep, acceleration, gyroscope and magnetometer readings, anthropomorphic and demographic data.

The performance metrics/data that can be analyzed and predicted can include velocity bands and associated distances, total steps, workout durations, and distances covered, power outputs, exercise loads, athletic speeds and stride lengths. Predictions may extend to heart performance metrics like stroke volume, cardiac output, and respiration rate, sodium, calcium, blood glucose, plasma volume, plasma electrolyte concentration, hematocrit, chloride, potassium, hemoglobin concentration, urine color, urine specific gravity, urine volume, urine osmolality, thirst, rate of perceived exertion, ear canal temperature, blood serum osmolality, and perceived hydration. The performance metrics can be tracked and generated by one or more of the sensors or additionally input manually by a user to the system for further analysis.

The system can be equipped with external devices, notably one or more mobile phones and specialized receivers/transceivers. This can be achieved either through wireless or wired connections. Data can be processed in real-time on the wearable device or can be transmitted to a backend server for more extensive analysis (onboard or offboard processing). The data can be used by the system and employ one or more advanced algorithms to predict the wearer's hydration status. Wearers are be alerted to their hydration levels via haptic feedback, auditory signals, or push notifications. The system provides a deep dive into an athlete's performance by analyzing and predicting metrics such as: velocity bands and associated distances, total steps, workout durations, and distances covered, power outputs (Joules or Calories), exercise loads (such as total, peak, and average loads per minute), athletic speeds and stride lengths.

Predictions may extend to heart performance metrics like stroke volume, cardiac output, and respiration rate, sodium, calcium, blood glucose, plasma volume, plasma electrolyte concentration, hematocrit, chloride, potassium, hemoglobin concentration, urine color, urine specific gravity, urine volume, urine osmolality, thirst, rate of perceived exertion, ear canal temperature, blood serum osmolality, body composition, and perceived hydration.

The system 100 can be paired with a mobile application 88, granting wearers/users direct access to their data and predictions on their smartphones. A web-based platform may also allow for a more in-depth analysis and is especially useful for athletic trainers, coaches, and other personnel to monitor individual athletes or an entire team's hydration status. The hydration status may be visualized as color coded from red, orange, to green metrics. The device may further comprise machine learning models for advanced predictions of hydration status among other predictive outcomes. The application can additionally communicate with a network that can store and further analyze the obtained data. The stored data can be used to track a user's progression and history.

The apparatus and system are configured to allow for the wearer to add more advanced sensors as needed. The apparatus can continuously calibrate itself to an on chip reference circuit and adjusts calibration as needed. The apparatus may also employ advanced algorithms that utilize machine learning from user-specific data over time to enhance prediction accuracy.

The apparatus comprises durable sensors and may also comprise waterproof coatings to protect the sensors from sweat and moisture. The sensors are configured to be coupled to various types of fabrics, and the apparatus is configured to allow for parts of the fabric that wear out over time to be replaced, without the need to dispose of the sensors.

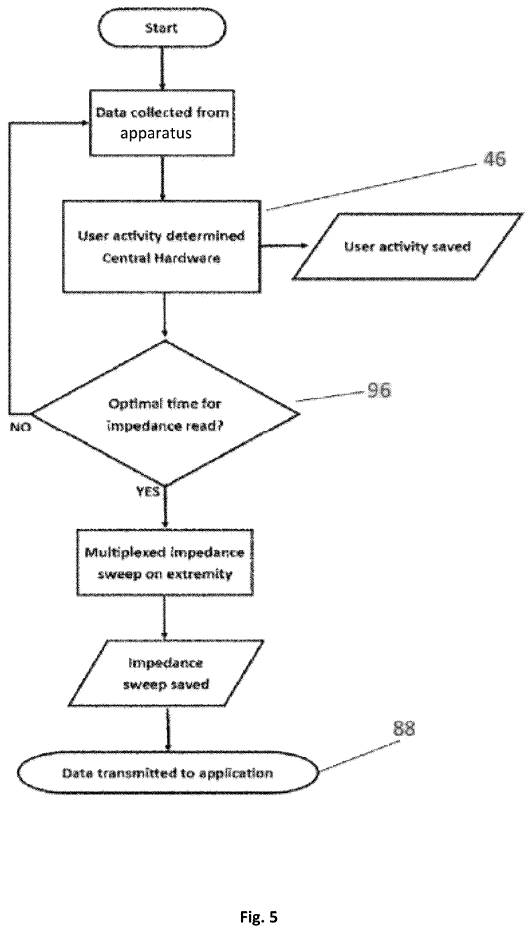

In one exemplary embodiment of the present disclosure, the apparatus can include a plurality of electrodes 10. The apparatus can include between about two and six electrodes, however, additional electrodes could potentially be implemented if desired. Two of the electrodes 10a,b can be current injecting 10a,b and the remainder electrodes 10 can be voltage sensing electrodes 10c,d,e,f as shown in FIGS. 1-4. Each of the electrodes 10 can have one or more ports that can be communicatively coupled to the processing means of the apparatus. As illustrated in FIG. 5, an exemplary embodiment of the apparatus of the present disclosure can further include the central hardware unit 46 comprises the central hardware 46, central enclosure 38, and textile-enclosure harness 58. The system 100 of the present disclosure can utilize dry textile electrodes for tetrapolar electrode configuration (TEC). These electrodes can be applied to the skin at a pressure of 15-40 mmHg, which is known to significantly decrease skin-electrode contact impedance.

The system can further include a wearable sensor network (WSN). In some exemplary embodiments, the electrodes can be included as part of the WSN. The WSN can further include other sensors for heart rate, acceleration, temperature, blood oxygen, electromyography (EMG), electrocardiogramhy (ECG), motions sensors, GPS sensors, sleep sensors, and anthropomorphic sensors, among others. The system can utilize circuitry to provide the functionality of the smart compression garment including the system 100 and the enclosures utilized to join the sensors to the smart compression garment. In one exemplary embodiment, the circuitry of the WSN comprises an accelerometer-gyroscope module network (AGMN), bio-impedance module 44, and a central hardware unit 46.

Connection, connector or adapter are envisioned as replacements for the harness. This central hardware unit 46 supplies power to all hardware, controls the AGMN 42 and the bio-impedance module 44, temporarily stores data, and transmits all data collected in raw, analyzed, encrypted, or other form to the mobile application. The central hardware 46 has several subsystems including an impedance converter 60 and network analyzer (bio-impedance module) 44, active analog filters 62, power management integrated circuits 64, multiplexed digital controls 66, a wireless transmission module 68, an analog to digital converter 70, a microcontroller 72, a peak detector 107, an internal clock 108, an SD card reader 109, a serial-to-USB bridge 110, a heart rate detector and a respiration monitor. The bio-impedance module 44 collects real and complex impedance data by delivering varying current magnitudes of up to 5 mA every 1 kHz across 5 kHz to 100 kHz s to the lower limb 74 and analyzing the delivered signal after it has passed through a section of tissue. A series of multiplexers 66 are utilized so that impedance analysis may be conducted on different segments of tissue by using different textile voltage-sensing electrodes 78 and current-injecting electrodes 80.

The current injection 80 and voltage sensing controls 78 result from active filters and multiplexers 76 that allow for different selection of stimulating current magnitude and sensing electrodes. All impedance measurements are taken via the tetrapolar electrode configuration 82 which minimizes the impact of variable skin-electrode contact impedances, polarization effects, and movement artifacts, since the voltage-sensing electrodes draw negligible current. These impedance measurements allow for a robust analysis of body composition of the lower limb, total body composition, swelling progression in the lower limbs, total body fluid retention, and patient compliance to compression therapy. Compliance is defined as when an individual is wearing the compression garment when instructed to by a medical professional. A battery 84 supplies the power required for all these functions and is rechargeable. Instead of a battery 84, a capacitor or other component capable of storing energy and providing power could be used. Instead of providing information on charge of the device to the user, the device could provide that information to any other individual or entity or could omit providing such information. The wireless transmission module 68 relays data to the mobile application 88. Instead of transmitting measured impedance values wirelessly, the device 20 could use a wired connection or other data relay mechanism to transmit measured values. The microcontroller controls all sensors and does minor data analysis.

Instead of utilizing the specific combination of hardware and software components listed here, other hardware and software components that allow for monitoring, transmission, recording, analyzing, or providing feedback on bio-impedance signals could be used. A temperature monitor or array of such monitors are also envisioned.

The mobile application 88 will receive data from the central hardware unit 46 and will further transmit this data for analysis to an online server. Instead of transmitting data to an application or a server, data could be kept locally on the device 20. Instead of performing data analysis remotely for example in an application or on a server, data analysis could be performed anywhere, for example locally on the device. This mobile application 88 will provide a certain amount of data analysis, display data to the user, provide the user with an understanding of the charge of the apparatus 100, and solicit information from the user 22 if necessary to complete the hydration status generation. Instead of providing data and/or feedback through an application, data and/or feedback could be provided directly on the device, for example though an LCD screen. Instead of providing data and/or feedback to the user, the application could provide data and/or feedback to an individual designated by the user 22, a healthcare professional, an insurance company, or any other party with a legitimate interest in the user's healthcare or activity data. The mobile application 88 is not required if the data-analyzing, data-recording, or feedback-providing functions of the device were incorporated into the device (central hardware unit 46 or similar).

The port nearest the middle of the hardware, and closest to the USB port, can be the current injecting port 12. The furthest electrode port from the USB port can be a voltage sensing pair of electrodes 10c,d that can be a positioned to approximate a first target position/area (Position A) 200 of a user 1. The port in between the current injection port and the Target A port 20 can be another voltage sensing pair of electrodes can be a second target position (Target B) 300. In some exemplary embodiments, the current injecting electrodes 10a,b are not the interior electrodes in the configuration and the first and second target positions may be oriented as illustrated in FIG. 1. This can ensure accuracy in the section of tissue 1 of a user that is being analyzing. In some exemplary embodiments, Target B can denote a particular configuration of current injecting and voltage sensing electrodes wherein the multiplexor in the apparatus selects the two innermost electrodes to be voltage sensing electrodes 10e,f. At other times, the various electrodes may be utilized for Target C 400 or Target D 300 configurations. The left side of FIG. 1 illustrates various Targets (A, B, C, D) areas and which voltage sensing electrodes are “active” for each configuration to be read by the apparatus.

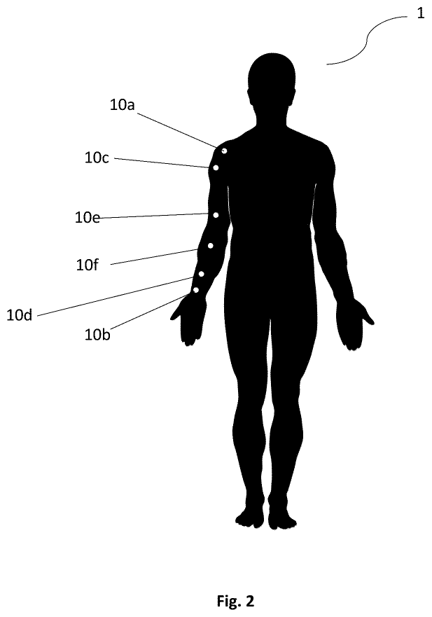

The interelectrode distance should also remain consistent. In some embodiments, the spacing of the current injecting electrode is about 2 inches away from the voltage sensing electrode of the same color. In some exemplary embodiments utilizing sensors to be placed on a user's arm, all electrodes should be placed in line with each other, generally on the lateral aspect of the arm (FIG. 2). Distance can be measured from the center of the snaps of the electrodes. The locations of the corresponding electrodes 10 can include a first wrist electrode placed about 1 inch proximally from the wrist joint and a second wrist electrode positioned about 2 inches proximally from the first wrist electrode. A first deltoid electrode can be positioned about 1 inch distally from the tip of the acromion and a second deltoid electrode can be positioned about 2 inches distally from the first deltoid electrode. An additional two electrodes can be utilized with a first arm electrode and a second arm electrode. The second electrode, first arm electrode, second arm electrode and second deltoid electrode can be positioned about equidistant from the next. The electrodes should ideally not be placed directly above a joint or bone as it may affect the accuracy of obtained data. The measured impedance is the section of tissue of a user 1 between two voltage sensing electrodes.

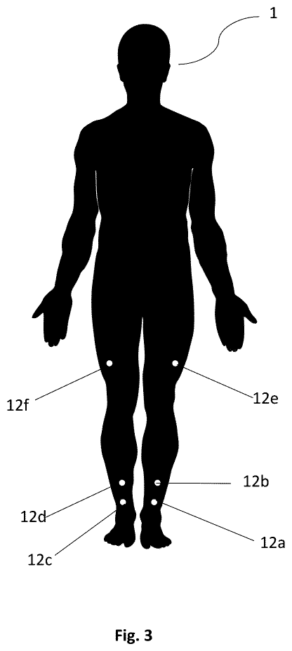

A circular current-injecting band electrode 12a,c can lie on each ankle and each upper thigh 12e,f. One or more other voltage-sensing electrodes 12b,d can lie on each leg between each pair of current-injecting band electrodes. A multiplexor may sequentially alternate which pair of voltage-sensing electrodes 12b,d is activated to enable segmental impedance analysis of each leg of a user. In some exemplary embodiments for a leg-to-leg comparison, for example, the current injecting electrodes would be about 1″ above the ankle on the medial aspect of the legs. Two inches above the center of the electrode (where the snap is located), we would place the voltage sensing electrodes for the first target (Target A) 200. The electrodes 12 can be generally symmetrically positioned along a median plane. A plurality of electrodes 12 could also be on the posterior, anterior or lateral aspect of the leg of a user 1 as shown in FIG. 3. It should be understood that the apparatus can use various numbers of electrodes and orientations.

One or more electrodes 12 can be placed in line with each other, on the lateral aspect of the leg. Distance can be measured from the center of the electrodes. A first ankle electrode 12a can be positioned about 1 inch proximally from the left leg's lateral malleolus. A second ankle electrode 12b can be positioned about 2 inches proximally from the first ankle electrode. A third ankle electrode 12c can be placed 1 inch proximally from the right leg's lateral malleolus. A fourth ankle electrode 12d can be positioned about 2 inches proximally from the third ankle electrode 12c. For the remaining two electrodes (a first thigh electrode 12e and a second thigh electrode 12f) can be equally spaced between the second ankle electrode and third ankle electrode. It should be understood that the distance between electrodes are approximate and various set pre-determined distances can be utilized based upon the anatomy of the user 1.

It should be understood that the electrodes can be placed on various locations of the user, including one or more limbs (leg(s) or arms(s)) as well as other locations on their tissue that provide a sufficient bio-impedance reading.

The electrodes of the device of the present disclosure can be configured to be alternately activated by the controller depending upon the configuration and position of the electrodes. A multiplexor may sequentially alternate which pair of voltage-sensing electrodes is activated to enable segmental impedance analysis of the analyzed area of the skin. Data can then be collected and analyzed from the plurality of the different locations of the electrodes located on a user. The collected data from each location of the sensing electrodes cab be sent to the processing unit for analysis to generate a hydrations status. Based upon the analysis and readings from the data, a signal can be transmitted to a display to alert a user of their hydration status.

The apparatus 100 of the present disclosure can be utilized to first establish and generate a hydration baseline from an individual based upon multiple factors. In one baseline factors can include elements such as demographics and an impedance profile. In some exemplary embodiments, the apparatus 100 can first measure the relative hydration of a subject in various pre-determined increments of time. In some embodiments, the pre-determined measurement points can include but are not limited to immediately before exercise, after exercising 45 min, and ˜4 hours after their exercise once they had rehydrated (drinking 2 liters of water). Once the hydration baseline is established the system can the continue to monitor a user by utilizing the plurality of electrodes and sensor data to determine a user's hydration.

In some exemplary embodiments, the impedance measured by the apparatus can be located at various positions between electrodes as previously illustrated in FIG. 1. The system can further include an artificial neural network (ANN). The system can utilize one or more algorithms to further optimize the accuracy of the hydration measurements. In one exemplary embodiment an ADAM optimization algorithm can be utilized. Additionally, variables can be calculated such as loss hydration loss calculated via binary cross entropy. The one or more algorithms and ANN can establish an improved modelling algorithm for monitoring hydration more accurately using bioimpedance. The system can then further utilize the raw measured data and optimized algorithm/module to generate a hydration status. The generated hydration status can be used to predict dehydration of a user.

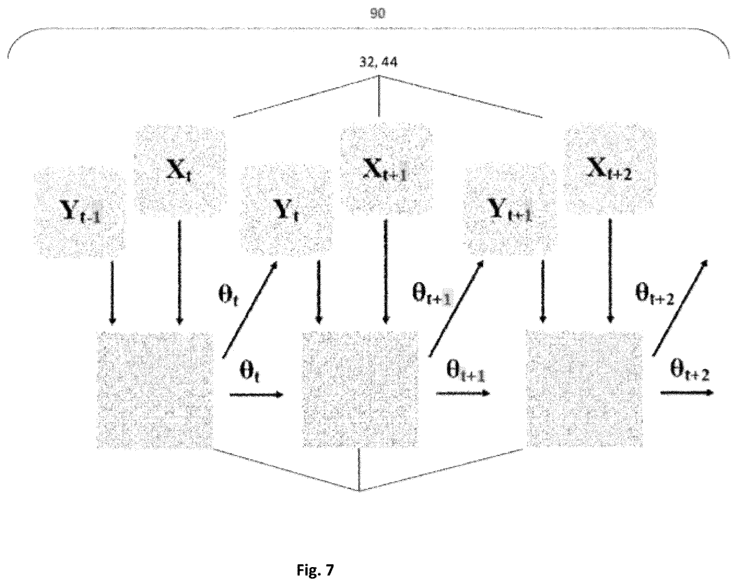

In some exemplary embodiments, the plurality of electrodes 10 can be implemented or coupled to one or more garments that allow the electrodes to come in contact with the user's tissue surface. The system 100 can take bio-impedance measurements in real-time, while a user 1 performs activities. The system 100 does not require a user 1 to remain unmoving. The system 100 can further utilize a Recurrent Neural Network (RNN) as shown in FIG. 7. To properly evaluate potential RNNs 30, the networks 34 may be trained and evaluated using open-source datasets, such as the University of Southern California human activity dataset (USC-HAD). Multiple architectures, such as Long Short-Term Memory Deep Recurrent Neural Networks (LSTM-DRNN) and LSTM Convolutional neural networks (LSTM CNN) may be evaluated.

The system 100 can fu

[symbol]

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. A system for detection of a hydration, wherein the system comprises:
a compression garment, the compression garment comprising a wearable sensor network and an application;
the wearable sensor network comprising a plurality of electrodes, wherein four electrodes of the plurality of electrodes are configured to be alternately activated in a limb of a user, wherein the four electrodes are configured to be activated in a configuration sequence consisting of two current-injecting electrodes and two voltage-sensing electrodes a first current injecting electrode, a first voltage sensing electrode, a second voltage sensing electrode, and a second current injecting electrode, respectively; and
the wearable sensor network comprising a multiplexor configured to sequentially alternate which two electrodes of the four electrodes are activated as the voltage-sensing electrodes, enabling a data collection and a data analysis from a plurality of different locations of the limb of the user,
the collected data comprising bio-impedance measurements of each location of the plurality of different locations of the limb of the user, and each location of the plurality of different locations of the limb of the user positioned between the two voltage-sensing electrodes,
wherein the wearable sensor network further comprises a central hardware unit, wherein at least one bio-impedance module are connected to the central hardware unit by wiring, textile electronic traces, or conductive connections;
the at least one bio-impedance module configured to collect the bio-impedance measurements from each location of the plurality of different locations of the limb of the user positioned between the two voltage-sensing electrodes, and
wherein the bio-impedance module is configured to collect the bio-impedance measurements by delivering a plurality of current signals to each location positioned between the two voltage-sensing electrodes of the limb of the user, the plurality of current signals having varying current magnitudes of up to 5 mA every 1 kHz across 5 kHz to 100 kHz; and
wherein the bio-impedance module is further configured to analyze the plurality of delivered current signals after the plurality of delivered current signals have passed through a section of tissue at each location positioned between the two voltage-sensing electrodes of each leg of the user.

2. The system of claim 1, further comprising the at least one accelerometer-gyroscope module configured to collect an additional data set from the user.

3. The system of claim 2, wherein the additional data set comprising acceleration and gyroscopic data from the limb of the user, wherein the central hardware unit is configured to supply power to the at least one accelerometer-gyroscope module and to the at least one bio-impedance module.

4. The system of claim 3, wherein the bio-impedance measurements from the bio-impedance module and the additional data set from the at least one accelerometer-gyroscope module are temporarily stored; and the bio-impedance measurements and the additional data set are transmitted to a mobile application.

5. The system of claim 4, wherein the mobile application is configured to receive a third data set from additional modules and to further transmit the bio-impedance measurements, the additional data set, and the third data set to an online server.

6. The system of claim 5, wherein the bio-impedance module can further include a third electrode placed in between the current injecting electrodes.

7. The system of claim 6, wherein the bio-impedance module can further include a fourth electrode placed in between the current injecting electrodes.

8. The system of claim 7, wherein the distance between the first voltage sensing electrode and the second voltage sensing electrode define a first target measurement area.

9. The system of claim 8, wherein the distance between the first voltage sensing electrode and the third voltage sensing electrode define a second target measurement area.

10. The system of claim 9, wherein the distance between the first voltage sensing electrode and the fourth voltage sensing electrode define a third target measurement area.

11. The system of claim 10, wherein the first target measurement area, second target measurement area, and third target measurement area provide impedance data to be processed.

12. The system of claim 11, wherein the distance between the second voltage sensing electrode and the fourth voltage sensing electrode define a fourth target measurement area.

13. The system of claim 12, wherein each of the one or more target measuring areas provide signal data to be processed and analyzed to determine a hydration status.

14. The system of claim 13, wherein the hydration status is communicated to the user through one or more transmission means.

15. The system of claim 14, wherein the transmission means comprises one or more of the following:
a visual display, haptic feedback, or an audio transmission.

16. The system of claim 15, wherein the compression garment is waist high stockings, knee high stockings, pantyhose, compression sleeve, headband, or any skin-contacting garment.

17. The system of claim 16, wherein the electrode configuration includes a circular current injecting band electrode on each end of the compression garment at the distal and proximal end of the garment.

18. The system of claim 17, wherein the voltage-sensing electrodes lie on the compression garment adjacent to the limb of the user between each pair of current-injecting band electrodes.

19. The system of claim 18, wherein the electrodes are configured to be applied to the skin at a pressure of 15-40 mmHg.

20. The system of claim 19, further comprising impedance monitor having a multiplexed, and a multi-frequency analyzer, which analyses several sections of tissue across the wearer in a sequence, wherein the hydration status is provided to a user in real-time, wherein the system can further generate a predictive output of future performance based on current hydration status.
